(19) United States
(12) Plant Patent Application Publication
(10) Pub. No.: US 2008/0010709 P1
(43) Pub. Date: Jan. 10, 2008

(54) Floribunda rose plant named 'HADICE'

(76) Inventor: Harvey Daniel Davidson, Orinda, CA (US)

Correspondence Address: HARVEY D. DAVIDSON, 3 EL VERANO RD., ORINDA, CA, 94563-1911, US

(21) Appl. No.: 11/481,389

(22) Filed: Jul. 6, 2006

(51) Int. Cl. A01H 5/00

(52) U.S. Cl. PLT/134

(57) ABSTRACT

A new and distinct rose cultivar of the Floribunda class, characterized by: Medium sized yellow flowers that fade to white; borne in many clusters of four to six flowers; very rapid repeat bloom; low bushy plants; stems with few prickles; some fragrance. The cultivar name of this plant is designated as ‘HADICE’. It is being marketed under the commercial name of ‘GOLDEN ICEBERG’.

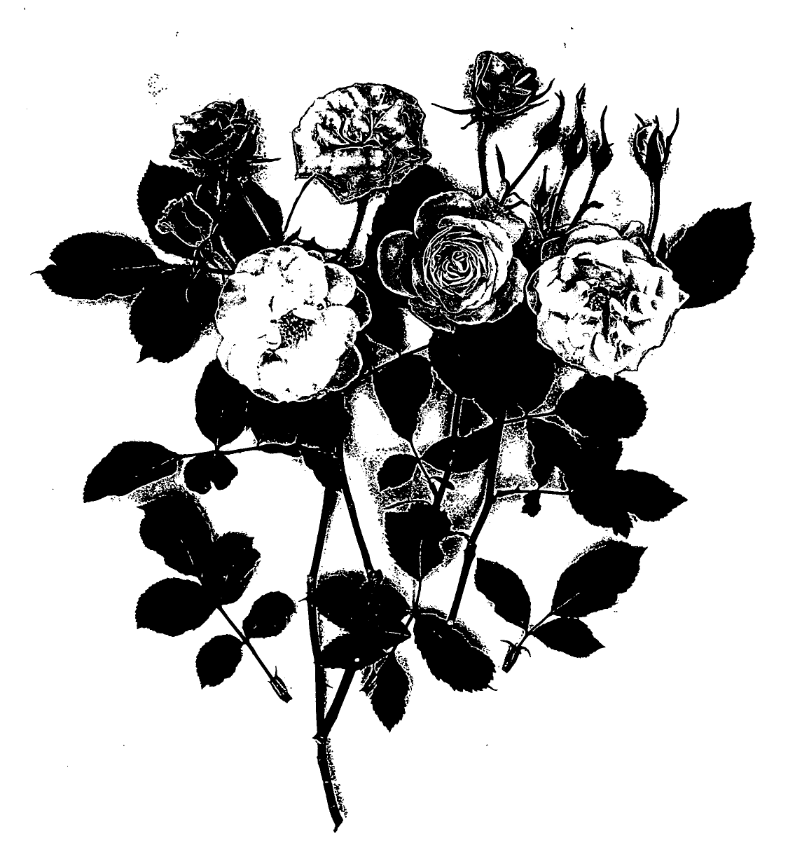

### BOTANICAL/COMMERCIAL CLASSIFICATION

Rosa hybrida/Hybrid Tea Rose Plant

### SPECIFICATION

The present invention relates to a new and distinctive cultivar of rose plant of the Floribunda class, which was originated by my crossing ‘SUNFLARE’ as the seed parent with ‘SUNFLARE’ as the pollen parent.

Among the novel characteristics possessed by the new cultivar which distinguish it from:
(a) All other cultivars of which I am aware are:
   (1) Forty to eighty blooms in initial and succeeding bloom cycles.
   (2) Eight centimeter yellow flowers fading to long lasting white.
   (3) Very rapid rebloom cycles, almost never without bloom.
   (4) Blooms in clusters of four to six flowers.
   (5) Extremely disease resistent.
(b) Seed parent: Yellow cluster of one to three flowers.
   Pollen parent: Same as seed parent.
      Asexual reproduction of the new rose cultivar by budding onto the rootstock ‘Dr. Huey’, as performed at Wasco. Calif., shows that the aforementioned and all other distinctive characteristics come true to form and are established and transmitted through succeeding propagations.

The accompanying drawing shows typical specimens of the vegetative growth and flowers of the new cultivar in different stages of development and as depicted in color as nearly true as it is reasonably possible to make the same in a color illustration of this character.

The following is a detailed description of my new cultivar, with color terminology in accordance with The Royal Horticultural Society Colour Chart (RHSCC). The terminology used in the color description herein refers to plate numbers such as: ‘Yellow Group 16A’ found in Fan 1 of The Royal Horticultural Society Colour Chart.
Parentage:
   Seed parent.—IRAR registered: ‘SUNFLARE’; Class: Florabunda. U.S. Pat. No. 5,248.
   Pollen parent.—Same as seed parent. The following observations are made of specimens grown outdoors in Orinda, Contra Costa County, Calif., in the month of October, 2005.

### FLOWER

Blooming habit:
   A. Bud.—(1) Size: Sepals enclosed, 19-22 mm from base of sepals to top of bud. (2) Form: Ovoid. (3) Sepals: Fully mature 25 to 35 mm in length; 10 to 14 mm extension beyond bud; no serration shown on sepal edges. Generally 3 out of 5 sepals show 5 to 6 appendages on each side, to 5 mm length. Sepals show glandular development on edges. Heavy cotton matting on inner surface, very fine matting on outer surface. Outside color: near to Yellow Green 144A; inside Greyed Green 194B. (4) Peduncle: Length — 4½ to 5½ cm. Surface: smooth, many fine hairs, few glandular hairs. Strength: firm. Color: near to Yellow Green 144A, no other shades. (5) Receptacle: Shape: oblong; 7-9 mm diameter; Color: same as peduncle. (6) Color: When sepals first divide, outside bud petals: near to Green Yellow 1C with slight shadings of 144D.
   B. Bloom.—(1) Size: When open: 7.5 to 8.5 cm. (2) Borne: Heavy cluster: 8 to 10 flowers per stem. (3) Form: Ovoid. (4) Petalage: Average — 28 to 32; center petaloids: average — 6 to 8. (5) Color: Early open: outer petals near Yellow 12C, inner petals near 12B. Aging petals: near Yellow 10D, base close to 12C.
   C. Petals.—(1) Texture: Moderately firm. (2) Appearance: Flexed, satiny. (3) Form: Symmetrically spaced. (4) Arrangement: Regular. (5) Persistance: Holds petals extremely well. (6) Fragrance: Moderately strong. (7) Lasting quality: On plant 15 to 20 days; cut 6 to 8 days.

### REPRODUCTIVE ORGANS

A. Stamen.—(1) Arrangement: Regular around styles. (2) Number: Average: 80-86. (3) Length: Average: 6 to 9 mm from base. (4) Filaments: Color: Close to Yellow Orange 15A. (5) Anthers: Color: Close to Yellow Orange 15A. (6) Pollen: Color: Close to Yellow Orange Group 17A.
B. Pistil.—(1) Styles: Length: 12-16 mm from ovary center; number 92-98; color above base of ovary: near to Red 46D; color internally in ovary: near to 157B, coated with white hairs. (2) Stigma: Color: Close to Yellow Orange 22A. (3) Ovary: Shape: Oblong 7-9 mm center cross section at sepal base.
C. Hips.—Does not set seed.

### PLANT

A. Form.—Spreading to upright.
B. Growth.—Very vigorous. Height: ¾ to 1 m; Width: average ¾ m.
C. Stem length.—Basal break to flower bud tip: average ¾ m.
D. Foliage.—Compound of 3 to 5 leaflets on bloom stem: 5 to 7 leaflets on main canes. (1) Mature size: 5 leaflet: tip of leaflet to stipule base: 15-16 cm, width: 10-11 cm. (2) Quantity: Stems well covered. (3) Color: New foliage: Upper side near Yellow Green 146A; under side near to 146B. Old foliage: Upper side near to Yellow Green 147A; under side near to Yellow Green 147B. (4) Shape: Oval with center pointed. (5) Texture: Very firm, very leathery. (6) Edge: Serration on outer edge: Average 4 points per 1 cm, with 1 to 2.5 mm peak. (7) Rachis: Glandular hairs only on upper side; very few prickles on lower side; length 1 mm. (8) Stipules: Length 2 to 2½ mm; auricle 5 to 7 mm. Edge of stipule to tip of auricle with many glandular hairs.
E. Resistance to disease.—Powdery mildew, rust and blackspot: Excellent.
F. Wood.—(1) New wood: Color close to Yellow Green 146B; Bark — smooth. (2) Old wood: Color close to Yellow Green Group 146A; Bark showing slight callusing on lower cane periphery shaded Greyed White 156C.
G. Prickles on mature wood.—few. (1) Size: 6-7 mm. (2) Number per 10 cm: Older wood 4-5 prickles; new wood none. (3) Shape: Very slight downward hook. (4) Color: Early stage close to Greyed Orange 176C; aging near 176A.
H. Winter hardiness.—Good.

I claim:

1. A new and distinct cultivar of the Floribunda class with essential features as follows:
1. Many blooms in long lasting overlapping cycles,
2. Medium sized double yellow flowers fading to white.
3. Flowers borne in heavy clusters.
4. Moderate fragrance.
5. Very rapid repeat bloom.
6. Bushy plants of medium height.
7. Few prickles, on lower canes only.
8. Extremely disease resistent.
Substantially as shown herein and described.
